(19) United States
(12) Patent Application Publication
(10) Pub. No.: US 2019/0144813 A1
(43) Pub. Date: May 16, 2019

(54) PARTICLES FOR USE IN ACOUSTIC STANDING WAVE PROCESSES

(71) Applicant: FloDesign Sonics, Inc., Wilbraham, MA (US)

(72) Inventors: Bart Lipkens, Bloomfield, CT (US); Krishna Kumar, Wilbraham, MA (US); Rui Tostoes, Northampton, MA (US); Thomas J Kennedy, III, Wilbraham, MA (US)

(21) Appl. No.: 16/208,512

(22) Filed: Dec. 3, 2018

Related U.S. Application Data
(63) Continuation-in-part of application No. 15/606,962, filed on May 26, 2017, now abandoned.
Application No. 15/606,962 is a division of application No. 15/053,359, filed on Feb. 25, 2016, now Pat. No. 9,663,756.
(60) Provisional application No. 62/182,009, filed on Jun. 19, 2015.
(60) Provisional application No. 62/612,979, filed on Jan. 2, 2018.
(60) Provisional application No. 62/621,585, filed on Jan. 24, 2018.

(51) Int. Cl. C12M 1/00; C12N 13/00; C12M 1/26

(52) U.S. Cl. CPC C12M 47/04; C12M 47/02; C12N 13/00; C12N 5/0062; C12M 33/08; C12N 2527/00; C12M 33/00

(57) ABSTRACT

Microparticles and nanoparticles made of various materials that are used in various configurations are disclosed. Such particles can also contain various types of materials as payloads to be used in the separation, segregation, differentiation, modification or filtration of a system or a host anatomy. The microparticles and nanoparticles are utilized in conjunction with an acoustic standing wave or an acoustic traveling wave in various processes.

### CROSS-REFERENCE TO RELATED APPLICATIONS

This application is a continuation-in-part of U.S. patent application Ser. No. 15/606,962, filed May 26, 2017, which is a divisional application of U.S. patent application Ser. No. 15/053,359, filed Feb. 25, 2016, which claims priority to U.S. Provisional Patent Application Ser. No. 62/182,009, filed Jun. 19, 2015. This application also claims priority to U.S. Provisional Patent Application Ser. No. 62/621,585, filed Jan. 24, 2018, and to U.S. Provisional Patent Application Ser. No. 62/612,979, filed Jan. 2, 2018. The above are all hereby fully incorporated herein by reference.

### BACKGROUND

The present disclosure relates to particles in the micrometer or nanometer range, which can be used with ultrasonically generated acoustic waves, including traveling and standing waves, to achieve trapping, concentration, and/or transport of the microparticles and nanoparticles to a target location.

Acoustophoresis is the separation of materials using acoustics, such as acoustic standing waves. Acoustic standing waves can exert forces on particles in a fluid when there is a differential in a parameter of the particles and fluid that can be influenced by acoustics, including density and/or compressibility, otherwise known as the acoustic contrast factor. The pressure profile in a standing wave contains areas of local minimum pressure amplitudes at standing wave nodes and local maxima at standing wave anti-nodes. Depending on their density and compressibility, the particles can be trapped at the nodes or anti-nodes of the standing wave. Generally, the higher the frequency of the standing wave, the smaller the particles that can be trapped.

At a micro scale, for example with structure dimensions on the order of micrometers, conventional acoustophoresis systems tend to use half or quarter wavelength acoustic chambers, which at frequencies of a few megahertz are typically less than a millimeter in thickness, and operate at very low flow rates (e.g., μL/min). Such systems are not scalable since they benefit from extremely low Reynolds number, laminar flow operation, and minimal fluid dynamic optimization.

At the macro-scale, planar acoustic standing waves have been used in separation processes. However, a single planar wave tends to trap the particles or secondary fluid such that separation from the primary fluid is achieved by turning off or removing the planar standing wave. Planar waves also tend to heat the media where the waves are propagated due to the energy dissipation into the fluid that is involved with generating a planar wave and the planar wave energy itself. The removal of the planar standing wave may hinder continuous operation. Also, the amount of power that is used to generate the acoustic planar standing wave tends to heat the primary fluid through waste energy, which may be disadvantageous for the material being processed.

### BRIEF DESCRIPTION

In various embodiments, methods are disclosed herein for moving particles within a host or primary fluid to a desired location using acoustic standing waves. The particles are placed within an acoustophoretic device, and an ultrasonic transducer is used to concentrate, trap, and/or move the particles as desired. The particles can also be used to interact or react with other particles or cells in the host or primary fluid. Sometimes, the structure of the particles can be changed upon exposure to the acoustic wave.

Disclosed herein in various embodiments are methods for concentrating particles in a primary fluid at a first location, comprising: flowing a fluid mixture comprising the particles and the primary fluid through an acoustophoretic device. The acoustophoretic device comprises: an acoustic chamber through which the fluid mixture flows; and an ultrasonic transducer including a piezoelectric material that can be driven to create an acoustic wave in the acoustic chamber. The ultrasonic transducer is driven to create the acoustic wave, thus concentrating the particles at the nodes and antinodes of the standing wave, with negative contrast factor to the anti-nodes and positive contrast factor materials accumulating at the nodes.

The acoustic wave can be a multi-dimensional acoustic standing wave, a planar acoustic standing wave, a combination of a multi-dimensional acoustic standing wave and a planar acoustic standing wave, or an acoustic traveling wave.

In some embodiments, the particles contain a payload. The payload can be a virus, a nucleic acid, a cytokine, a pharmaceutical molecule, a liquid, a gas, or mixtures thereof. After moving the particles to the first location, the payload can be released.

The particles can be microparticles or nanoparticles. The particles may be solid, cellular, hollow, multilayer or a foam.

The particles can be made of one or more polymeric materials, ionomers, ceramics, or glass. Examples of polymeric materials include polyethylene, polypropylene, polystyrene, divinylbenzene, poly methyl methacrylate, polysaccharide, polylactic acid (PLA), and poly(lactic-co-glycolic acid) (PLGA).

Particles may also be produced from agarose and polyhyaluronic acid. These particles will dissolve in vivo, thus causing no deleterious issues with the patient.

The particles can be formed from multiple layers of polymeric materials. In some embodiments, the particles may be hollow, made of glass, and/or have an ablative polymer coating an exterior surface of the glass. The ablative polymer may be a polysaccharide that is functionalized with an antigen, antibody, or protein.

In other embodiments, the particles comprise: a liquid core; and a lipid shell encapsulating the liquid core. The liquid in the liquid core may comprise a perfluorocarbon. The perfluorocarbon may be perfluoropentane, perfluorohexane, perfluorooctane, perfluorooctyl bromide, perfluorodichlorooctane, or perfluorodecalin.

The lipid shell can be formed from dipalmitoylphosphatidylcholine (DPPC), 1,2-palmitoyl-phosphatidic acid (DPPA), a lipid-polyethylene glycol conjugate, or a complex of a lipid with albumin. The lipid shell can be functionalized with streptavidin, biotin, avidin, or an antibody.

Also disclosed herein are particles, comprising: a liquid core; and a lipid shell encapsulating the liquid core.

The liquid in the liquid core may comprise a perfluorocarbon. The perfluorocarbon may be perfluoropentane, perfluorohexane, perfluorooctane, perfluorooctyl bromide, perfluorodichlorooctane, or perfluorodecalin. The lipid shell may be formed from dipalmitoylphosphatidylcholine (DPPC), 1,2-palmitoyl-phosphatidic acid (DPPA), a lipid-polyethylene glycol conjugate, or a complex of a lipid with albumin. The lipid shell can be functionalized with streptavidin, biotin, avidin, or an antibody.

A process known as acoustic droplet vaporization (ADV) can be used to generate a phase shift of the liquid core of such particles from liquid to gas using an acoustic wave. The vapor pressure of the liquid is a function of temperature, and is not necessarily based upon the liquid chemistry. Any liquid that has a normal boiling point near or below the body temperature can be used for these processes. Fluorocarbons may be utilized in these processes because of their low toxicity and high contrast factor.

A spacer may be placed in between the particle and the antigen, antibody, or protein. The spacer is typically a polyethylene glycol (PEG) molecule that allows for less charged interference from the particle when materials are binding to the functionalized molecule on the surface of the particle.

These materials may also be utilized for the transduction of cells, for example by sonoporation. The bubbles are acoustically cavitated near a cell wall and create oscillations that contribute to opening a passage in the cell wall. Collapsing bubbles via acoustically induced cavitation can produce jets of fluid that contribute to opening cell walls.

In another configuration, these bubbles can contain a therapeutic agent. Thus, when the bubbles are broken via the acoustic excitement, the jetting material is a therapeutic and enters the cell during this process. The therapeutic can be a small molecule, a large molecule, or a piece of genetic material utilized in modifying the DNA of the target cell.

More generally, the particles described herein can be used as an agent to cause a change to a second material when the particles are impinged upon by acoustic waves. For example, the particles may be used to increase the contrast factor of the second material, which is a factor in increasing acoustophoretic efficiency. As another example, liquids may be delivered by the particles to cause changes to cell barriers in operations such as sonoporation.

Also discussed herein are techniques and devices for generating material clusters that can be used to improve gravity or buoyancy separation and collection efficiency of the materials. Improved, continuous, acoustophoresis devices using improved fluid dynamics are also discussed, as well as control of the devices for desired performance. The materials can be preferentially trapped in or released from/through the acoustic wave, depending on various parameters and characteristics of the acoustic wave and/or materials, including, for example, the contrast factor of the material.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a micrograph of particles in accordance with the present disclosure.

FIG. 2A is a Scanning Electron Microscope (SEM) photograph of a solid particle.

FIG. 2B is an SEM photograph of a cellular particle.

FIG. 2C is a micrograph of a hollow particle.

FIG. 2D is an illustration of a particle having a solid core and an exterior layer.

FIG. 2E is an illustration of a hollow particle having material within the core, and an exterior layer that can be ablated to release the material within the core.

FIG. 2F is an illustration of a hollow particle having a payload, and a shell surrounding the payload.

FIG. 3 is a schematic illustration of a particle comprising a liquid core and a lipid shell.

FIG. 4 is a schematic illustration of several particles being aligned/grouped with each other.

FIG. 5A is a graph showing number of particles versus particle diameter for diameters of 0.6 microns to 1.25 microns, for initial droplets and droplets after incubation with NeutrAvidin®. The y-axis is linear and runs from 0 to 3.0×10⁸ at intervals of 1.0×10⁸. The x-axis is in microns, and runs from 0.6 to 1.2 at intervals of 0.2.

FIG. 5B is a graph showing number of particles versus particle diameter for diameters of 1.25 microns to 2.25 microns, for initial droplets and droplets after incubation with NeutrAvidin®. The y-axis is linear and runs from 0 to 8.0×10⁶ at intervals of 2.0×10⁶. The x-axis is in microns, and runs from 1.4 to 2.2 at intervals of 0.2.

FIG. 5B is a graph illustrating droplet size distribution in accordance with the present disclosure.

FIG. 6 illustrates a process for preparing particles that contain a payload, and the subsequent release of that payload, in accordance with the present disclosure.

FIG. 7 is a depiction of a traveling wave in accordance with the present disclosure.

FIG. 8 is a depiction of a standing wave in accordance with the present disclosure.

FIG. 9 is a front cross-sectional view of an acoustophoretic device in which the methods of the present disclosure can be used.

FIG. 10 is an exterior perspective view of the acoustophoretic device of FIG. 9.

FIG. 11 is a cross-sectional diagram of an ultrasonic transducer of the present disclosure. An air gap is present within the transducer, and no backing layer or wear plate are present.

FIG. 12 is a cross-sectional diagram of another ultrasonic transducer suitable for use in the present disclosure. An air gap is present within the transducer, and a backing layer and wear plate are present.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/components/steps and permit the presence of other ingredients/components/steps. However, such description should be construed as also describing compositions, articles, or processes as “consisting of” and “consisting essentially of” the enumerated ingredients/components/steps, which allows the presence of only the named ingredients/components/steps, along with any impurities that might result therefrom, and excludes other ingredients/components/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The term “about” can be used to include any numerical value that can vary without changing the basic function of that value. When used with a range, “about” also discloses the range defined by the absolute values of the two endpoints, e.g. “about 2 to about 4” also discloses the range “from 2 to 4.” The term “about” may refer to plus or minus 10% of the indicated number.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

It should be noted that many of the terms used herein are relative terms. For example, the terms “upper” and “lower” are relative to each other in location, e.g. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, e.g. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, e.g. ground level. However, these terms should not be construed to require structures to be absolutely parallel or absolutely perpendicular to each other. For example, a first vertical structure and a second vertical structure are not necessarily parallel to each other. The terms “top” and “bottom” or “base” are used to refer to surfaces where the top is always higher than the bottom/base relative to an absolute reference, e.g. the surface of the earth. The terms “upwards” and “downwards” are also relative to an absolute reference; upwards is always against the gravity of the earth.

The present application refers to “the same order of magnitude.” Two numbers are of the same order of magnitude if the quotient of the larger number divided by the smaller number is a value of at least 1 and less than 10.

The term “virus” refers to an infectious agent that can only replicate inside another living cell, and otherwise exists in the form of a virion formed from a capsid that surrounds and contains DNA or RNA, and in some cases a lipid envelope surrounding the capsid.

The term “crystal” refers to a single crystal or polycrystalline material that is used as a piezoelectric material.

The present disclosure refers to “microparticles.” This term refers to particles having an average particle diameter of 1 micrometer (μm) to 1000 μm.

The present disclosure refers to “nanoparticles.” This term refers to particles having an average particle diameter of 1 nanometer (nm) to less than 1000 nm.

Some of the materials discussed herein are described as having an average particle diameter. The average particle diameter is defined as the particle diameter at which a cumulative percentage of 50% (by volume) of the total number of particles are attained. In other words, 50% of the particles have a diameter above the average particle size, and 50% of the particles have a diameter below the average particle size. The size distribution of the particles may include a Gaussian distribution, with upper and lower quartiles at 25% and 75% of the stated average particle size, and all particles being less than 150% of the stated average particle size. Any other type of distribution may be provided or used. It is noted that the particles do not have to be spherical. For non-spherical particles, the particle diameter is the diameter of a spherical particle having the same volume as the non-spherical particle.

Particles may be described herein as having a “core” and “shell” structure. In such particles, the core will be made of a liquid or gas, and the shell will be made of one or more layers of a relatively solid material (relative to the core). The shell and the core can be distinguished by their phase of matter. The term “particle” is meant to refer to any type of individual structure that may be suspended in a fluid such as a liquid or gas and may be in any phase, e.g., solid, liquid or gas and combinations thereof.

“Organic” and “inorganic” materials are referred to herein. For purposes of the present disclosure, an “organic” material is made up of carbon atoms (often with other atoms), whereas an “inorganic” material does not contain carbon atoms.

The present disclosure may refer to temperatures for certain process steps. In the present disclosure, the temperature usually refers to the temperature attained by the material that is referenced, rather than the temperature at which the heat source (e.g. furnace, oven) is set. The term “room temperature” refers to a range of from 68° F. (20° C.) to 77° F. (25° C.).

The present disclosure relates to particles that are used in conjunction with acoustophoretic devices. The acoustophoretic device generates acoustic waves that can be used in various ways. For example, the acoustic waves can be used to move the particles to a desired location, or to change certain properties of the particles, or to enhance reaction of the particles with other particles (such as biological cells). The particles can be microparticles or nanoparticles, as desired. The particles will first be discussed herein, then the acoustophoretic devices themselves. Various methods and reactions that can be performed using the particles with the acoustophoretic devices will also be discussed.

Particles

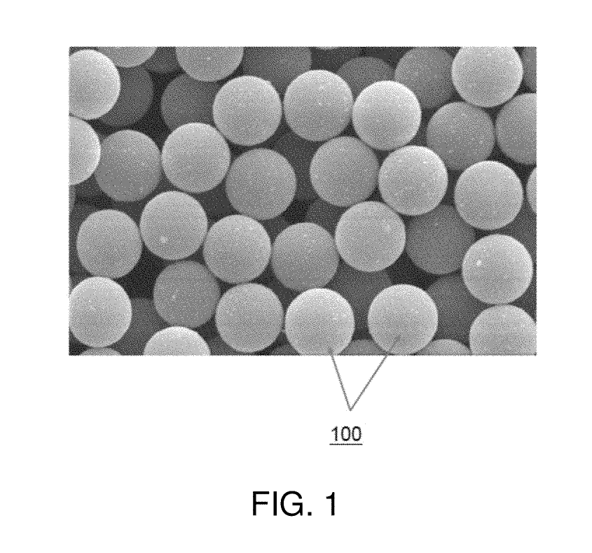

As discussed above, the particles are generally microparticles or nanoparticles. The particles may be spherical in shape, as shown in FIG. 1 with reference numeral 100. However, their shape can vary. For example, the particles could be ellipsoidal or elongated along a longitudinal axis.

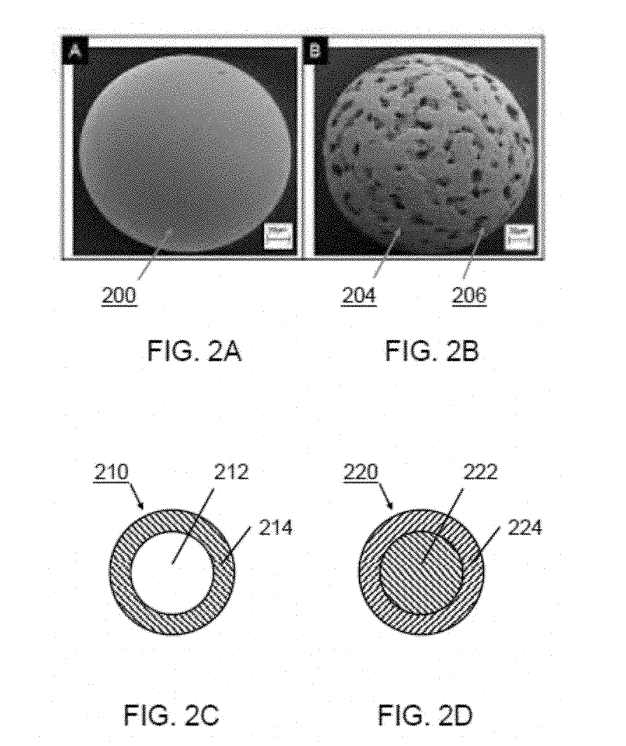

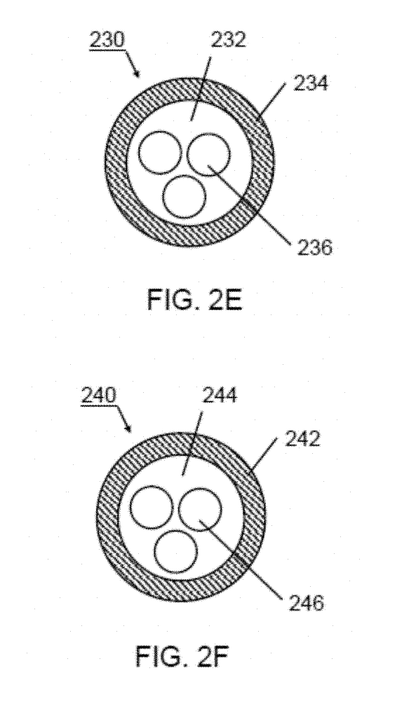

The particles may be, for example, solid, cellular, hollow, or a foam. A solid particle does not contain any voids or cavities, and a solid particle 200 is illustrated in FIG. 2A. A cellular particle contains voids/cavities in its interior, and has passages from the exterior of the particle to those voids/cavities (analogous to an open-cell foam). A cellular particle 204 is illustrated in FIG. 2B, with voids/cavities 206 visible from the exterior. A hollow particle is illustrated in FIG. 2C. The hollow particle 210 has one or more large voids or cavities 212 within a solid exterior surface 214. A foam contains multiple voids/cavities, each void being completely surrounded by solid material (also known as a closed-cell foam).

In particular embodiments, the particles may be made of inorganic materials, organic materials, or combinations thereof. Such materials may include polymers, ionomers, ceramics, glass, and other materials.

Polymers that may be utilized for the manufacture of the particles discussed herein include polyolefins such as polyethylene and polypropylene. The polyethylene may be a linear low density polyethylene, a high density polyethylene, a low density polyethylene, or an ultra-high molecular weight polyethylene. The polyethylene or polypropylene materials may be polymerized with a catalyst such as a peroxide catalyst, a Ziegler-Natta catalyst or a metallocene catalyst.

Other polymers that may be utilized in manufacture of the particles include polystyrene, divinyl benzene, polymethyl methacrylate (PMMA), polysaccharides such as agarose and agar, poly lactic acid (PLA), and poly(lactic-co-glycolic acid) (PLGA).

These polymers may be utilized to make up the bulk of the particles. microparticles or nanoparticles. The polymers may also be utilized in various combinations to make particles out of multiple layers (e.g. multilayer particles). Different polymers can be used to obtain the desired effect for the particles. For example, making particles out of multiple different layers can be used to obtain both a desired density and a desired acoustic contrast factor, or to obtain a desired behavior or interaction for the particle.

As one example, a polystyrene bead may be created in an aqueous suspension and then freeze-dried to obtain a foam particle. When the freeze-dried foam particle is suspended in water, small bubbles may form on its surface, resulting in a foam particle with a relatively solid core and nano-bubbles trapped in cavities on the surface of the foam particle.

As another example, a polymethyl methacrylate core may be coated with a PLA or PLGA polymer that forms an exterior surface for specialized drug delivery or interaction with biological cells. The resulting particle may be considered a solid particle or a foam particle (depending on the construction of the polymethyl methacrylate core), and may have a negative or positive contrast factor depending upon the density of the composite particle and the speed of sound in the composite particle. This example is illustrated in FIG. 2D. The particle 220 has a PMMA core 222 with a PLA or PLGA coating 224.

As another example, the exterior layer of the particle may be useful for causing biological interaction/reaction of the particle. For example, the exterior layer may permit the particle to be used for affinity binding. As another example, the particle could be a hollow particle with an exterior layer that is made from an ablative material (e.g. a material that melts or dissolves). This structure would permit materials held in the core of the hollow particle to be released after a certain period of time or exposure to sufficient heat or other energy, which would permit the particles to travel to a desired target or location. This example is illustrated in FIG. 2E. The particle 230 has a core 232 with an exterior layer 234 made of the ablative material. Material 236 is present within the core.

In some embodiments, the acoustic contrast factor of the particle can be changed. For example, hollow glass particles could be coated with an ablative polymer, such as a polysaccharide that is functionalized with antigens or antibodies or other protein or biological moieties. The particles could begin a process with a first acoustic contrast factor, and then be changed to a second acoustic contrast factor by removal of the ablative polymer.

In some embodiments, the particles of the present disclosure have a positive acoustic contrast factor. Such particles can be trapped at the nodes of an acoustic standing wave. In other embodiments, the particles of the present disclosure have a negative acoustic contrast factor. These particles will be trapped at the anti-nodes of an acoustic standing wave. If the particle changes in the acoustic contrast factor while in a processing system or in vivo, the particle could then migrate from a node to an anti-node if the particle changes from a positive contrast factor to a negative contrast factor and vice versa if a particle changes from a negative contrast factor to a positive contrast factor.

In some embodiments, the particles of the present disclosure contain a payload. The payload may include a primary, secondary, tertiary and/or more materials that are delivered by the particles to a specific area or cell population. Examples of materials that can be delivered as a payload include a virus, a nucleic acid, a cytokine (such as an interleukin), a pharmaceutical molecule, a liquid, or a gas, or mixtures of such materials. These payloads can be delivered to a desired target or location (by acoustic co-location) and then release the payload. That the payload would affect a target at the desired location, for example causing a change in the morphology, biochemistry or other attribute of the targeted material. This example is illustrated in FIG. 2F. The particle 240 is hollow, with a solid shell 242 surrounding a core 244 that contains a payload 246.

In addition, the particles of the present disclosure could also be affected by an outside force such as a magnetic, electromagnetic, dielectric, ultrasonic or other type of energy. By affecting the particles with an outside energy source, the particles may be activated upon reaching certain process steps (e.g. an affinity binding) or a specific part of a host's anatomy (e.g. to destroy a tumor located within a patient's body).

In some further embodiments, the particles are of a core-shell structure, with a liquid core encapsulated by a lipid shell. In more particular embodiments, the liquid in the liquid core is a perfluorocarbon (PFC). The term “perfluorocarbon”, as used in the present disclosure, refers to molecules in which all of the hydrogen atoms have been replaced with a halogen, and a majority of the halogen atoms are fluorine atoms. For purposes of the present disclosure, “halogen” refers to fluorine, chlorine, and bromine. Specific examples of PFCs include perfluoropentane (PFP), perfluorohexane (PFH), perfluorooctane (PFO), perfluorooctyl bromide (PFOB, C₈F₁₇Br), perfluorodichlorooctane (PFDCO, C₈F₁₆Cl₂), or perfluorodecalin (PFD, C₁₀F₁₈).

These PFC liquids have unique properties. The PFC liquids are denser than water, have low surface tension and have low viscosity. The PFC liquids also have a high capacity to absorb oxygen and nitrogen. Perfluorocarbon liquids have a low speed of sound, are highly chemically inert, and are biocompatible. Table 1, below, shows various physical and acoustic properties of various PFC liquids which may be used in particles, along with other polymers for comparison. It is noted that the compressibility of the PFC liquids is very high compared to biological cells.

TABLE 1

| | | Speed of | Boiling | | Specific | Surface | |
|---|---|---|---|---|---|---|---|
| | Density | Sound | Point | Contrast | Gravity | Tension | |
| Compound | (kg/m³) | (m/s) | (° C.) | Factor | (g/mL) | (mN/m) | Compressibility |
| PFP | 1600 | 477 | 29 | −1.59 | 1.6 | 9 | 27.46 × 10¹⁰ |
| (Perfluoro | | | | | | | |
| pentane) | | | | | | | |
| PFH | 1670 | 548 | 57 | −1.44 | 1.63 | 12 | 19.93 × 10¹⁰ |
| (Perfluoro | | | | | | | |
| hexane) | | | | | | | |
| PFOB | 1920 | 630 | 141 | −0.55 | 1.9 | 16 | 13.12 × 10¹⁰ |
| (Perfluoro | | | | | | | |
| octyl | | | | | | | |
| bromide) | | | | | | | |
| PMMA | | 2700 | | 0.299 | 1.18 | | |
| Polystyrene | | 2350 | | 0.22 | 1.06 | | |
| Cell | 1060 | 1600 | | | | | 3.68 × 10¹⁰ |

Specific examples of lipids that can be used to form the lipid shell include dipalmitoylphosphatidylcholine (DPPC), 1,2-palmitoyl-phosphatidic acid (DPPA), 1,2-dipalmitoyl-sn-glycero-3-phosphoethanolamine (DPPE), and 1,2-distearoyl-sn-glycero-3-phosphoethanolamine (DSPE). These lipids can also be used in a lipid-polyethylene glycol conjugate, or a complex of a lipid with albumin (such as bovine serum albumin or human serum albumin). The lipid shell can be functionalized with streptavidin, biotin, avidin, an antibody, or other functionalized moieties.

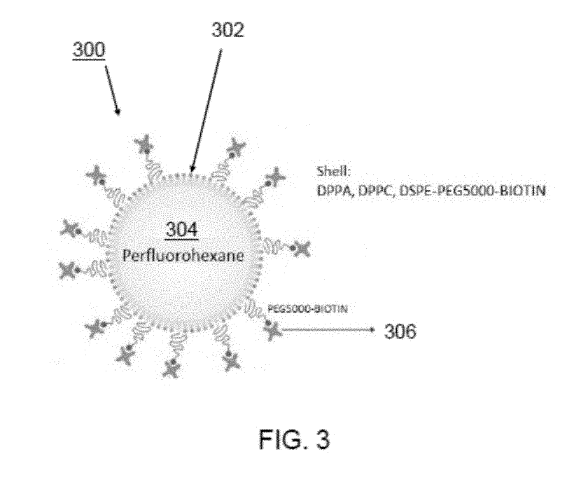

This structure is illustrated in FIG. 3. The particle 300 is made of a lipid shell 302 that surrounds a liquid core 304, in this example perfluorohexane. The shell can be made of DPPA, DPPC, or a functionalized lipid-glycol conjugate, here labeled as DSPE-PEG5000-BIOTIN. Also illustrated is an avidin derivative 306 that binds to the biotin of the lipid shell.

The lipid shell is used to attach the particle to another molecule, and for protection of the liquid core. These lipid-PFC particles are believed to be able to produce transient changes in the permeability of cell membranes after ultrasonic-induced cavitation while reducing cellular damage. They may enable tissue-specific or site-specific intracellular delivery of genetic materials, both in vitro and in vivo. They can be used to enhance the efficacy of gene delivery, for use as a non-viral vector system.

Generally, a PFC liquid and a lipid solution are combined to make a liquid core with a lipid shell. The PFC liquid is dispersed in another solution to form droplets. An emulsifier may be added to the solution, to prevent the droplets from coalescing. In some embodiments, phospholipids are used as the emulsifier/surfactant. A PFC liquid is dispersed by different methods depending upon the size of droplets desired for the application. To create small nanometer-sized droplets, ultrasonic agitation may be used. To create larger droplets, a vial shaker may be used to agitate the liquid mixture.

In some embodiments, a lipid solution consists of several different lipid materials in solution. The procured lipids are stored in a freezer at about −20° C. At this temperature, the lipids are in a solid state. The lipids may be taken out of the freezer and left at room temperature for about 20 minutes before use. This is done to bring the lipids to gel state. Since lipids generally do not dissolve in water, propylene glycol may be used to dissolve them. It is desirable to not dissolve all the lipids at once in the propylene glycol, as putting all the lipids at a time may result into formation of white clumps in the solution. The solubility of the each lipid material should be compared and the lipid material with maximum solubility should be dissolved first in the propylene glycol and so on. Since, the solubility of the lipids are a function of temperature of the solution, the solution should be maintained at a temperature above the transition temperature of the lipids. Table 2 is an example of a lipid composition.

TABLE 2
Lipids

| | Total lipid concentration | | | |
|---|---|---|---|---|
| | 1 mg/ml | | | |
| | | | Avanti | |
| | | Molar | catalog | No of |
| | Mol wt (gm) | ratio | information | carbons |
| DPPA | 670.87 | 11 | | 16 |
| DPPC | 734.04 | 82 | | 16 |
| DPPE-PEG-5000 | 5744 | 0 | 880200 | 16 |
| DSPE-PEG-2000 | 2805.49 | 0 | 880120 | 18 |
| DSPE-PEG-2000- | 3070 | 0 | 880129 | 18 |
| BIOTIN | | | | |
| DSPE-PEG-5000- | 5670 | 7 | | 18 |
| BIOTIN | | | | |

| V (stock lipid | | | DSPE-PEG-5000- |
|---|---|---|---|
| volume), mL | DPPA (mg) | DPPC (mg) | BIOTIN (mg) |
| 10 | 0.69 | 5.61 | 3.70 |
| 20 | 1.38 | 11.22 | 7.40 |
| 30 | 2.06 | 16.84 | 11.10 |
| 40 | 2.75 | 22.45 | 14.80 |
| 50 | 3.44 | 28.06 | 18.50 |
| 60 | 4.13 | 33.67 | 22.20 |
| 70 | 4.82 | 39.28 | 25.90 |
| 80 | 5.50 | 44.89 | 29.60 |
| 90 | 6.19 | 50.51 | 33.30 |
| 100 | 6.88 | 56.12 | 37.00 |
| 110 | 7.57 | 61.73 | 40.70 |
| 120 | 8.26 | 67.34 | 44.40 |

One representative process for creating a lipid solution is as follows. First, the propylene glycol is heated to the maximum transition temperature of the lipid blend for mixing. Next, the lipid material with maximum solubility is added to the heated propylene glycol. The lipid material and propylene glycol are then mixed in a bath sonicator. Sequentially, lipids of lower solubility are added into the propylene glycol mixture while in the bath sonicator.

A mixture of glycerol and buffer solution may be prepared simultaneously. The glycerol and buffer solution is heated to the maximum transition temperature. Once the lipid-propylene glycol solution is translucent (free of white clumps) in the sonicator, the lipid-glycol solution is mixed with the glycerol-buffer solution. The resulting mixture is homogenized with a homogenizer operating at 3000 rpm. The homogenization is performed for about one hour. During the homogenization process, the temperature is maintained at the maximum transition temperature of the lipids.

The prepared lipid solution is filtered to remove any possible contaminants such as dust, undissolved lipid clumps, etc. The filtering process may be performed with a hydrophilic syringe filer. The filters are soaked in the same temperature batch prior to use. In some embodiments, a 2.0 micron filter is used. In other embodiments, a 0.8 micron filter is used. In yet other embodiments, a 0.45 micron filter is used. In some embodiments, a combination of filters may be used.

The lipid solution is then mixed with the PFC liquid in a narrow vessel to create core-shell particles. The PFC liquid is first placed into a vessel and the lipid solution is poured on top. To make smaller sized droplets, the amount of PFC liquid in the vessel should be minimal. As the ratio of PFC liquid volume to lipid solution volume increases, the size of the formed droplet increases until it reaches a plateau for a given sonication power. It is to be noted that the PFC liquids are low strength as they have low surface tension values. Therefore, the sonication amplitude should be selected appropriately and the input of ultrasonic waves should be done in a pulsed mode rather than in a continuous mode. The tip of a horn sonicator assembly should be placed at the interface of two liquid solutions. To avoid formation of bubbles/foam the horn should be sufficiently inside the solution. Here, the aim is to prepare a droplet solution, so the narrow vessel is submerged in a transparent low temperature bath. The transparent low temperature bath is made, for example, by making a supersaturated solution of salt and then storing the salt solution in the freezer at −20° C. The sonication produces smaller beads.

In one example, the lipid solution may comprise about 1 mL propylene glycol+1 mL glycerol+8 mL buffer solution+lipid blend of 10 mg. 9 mL of the lipid solution may be combined with about 1 mL of PFC solution. The Lipid-PFC solution may be sonicated. For a 0.5 inch probe and 750 watt sonicator, a PFC solution utilizing 30% PFP is sonicated for about 3 seconds on and about 10 seconds off until a total sonication time of about 15 seconds is reached. A PFC solution utilizing 40% PFH is sonicated for about 3 seconds on and about 10 seconds off until a total sonication time of about 15 seconds is reached. A PFC solution utilizing 50% PFOB is sonicated for about 3 seconds on and about 10 seconds off until a total sonication time of about 15 seconds is reached. The sonication produces a droplet solution.

To prepare larger sized droplets, the quantity of PFC liquid is increased and the power input of the sonicator is reduced drastically. In another non-limiting exemplary embodiment, 500 microliters of PFC and 2 mL of lipid solution may be placed in a 3 mL vial. The vial may then be shaken in a vial mixer at 4800 rpm for 30 seconds. The prepared droplet suspension may have some microbubbles. In cases where microbubbles are present, the solution may be centrifuged.

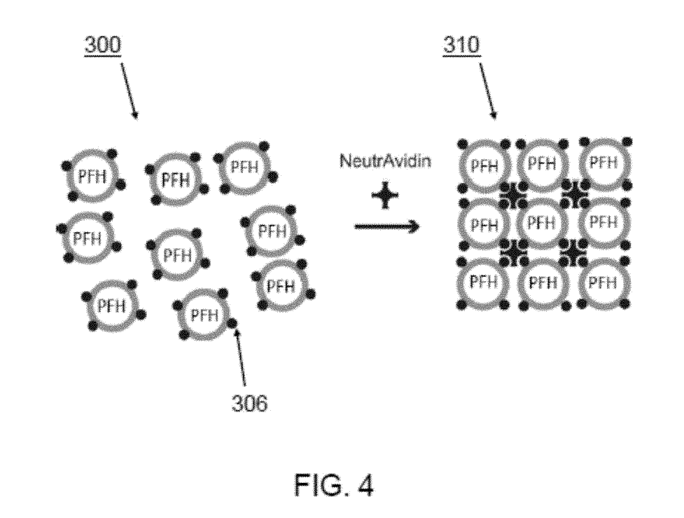

The binding efficiency of these PFC-lipid particles can be tested by adding NeutrAvidin® to the droplet solution. NeutrAvidin® is a deglycosylated version of avidin, with a mass of approximately 60,000 daltons. Like avidin itself, NeutrAvidin® is a tetramer with a strong affinity for biotin (Kd=10-15 M). Because the carbohydrates are removed, though, undesired lectin binding is reduced to undetectable levels, yet biotin binding affinity is retained. NeutrAvidin® also has a near-neutral pI (pH 6.3), minimizing non-specific interactions with the negatively-charged cell surface or with DNA/RNA. Neutravidin® still has lysine residues that remain available for derivatization or conjugation. Alternatively, If a binding complex is present (e.g. avidin-biotin), aggregates may be formed. This aggregation phenomenon may be one way to skew the droplet population towards a larger size. This mechanism is illustrated in FIG. 4. Nine PFC-lipid particles 300 are illustrated on the left-hand side, with the lipid shell surrounding the liquid PFH core. The lipids include a biotin complex 306. Upon exposure to avidin or similar molecule, the particles aggregate into a larger particle 310.

In one experiment, 5 mL of the droplet solution was taken and incubated with 100 microliters of 5 mg/mL NeutrAvidin® solution. This combination solution was left for one hour, and size measurement was done from the original droplet solution and from the droplet solution incubated with NeutrAvidin®.

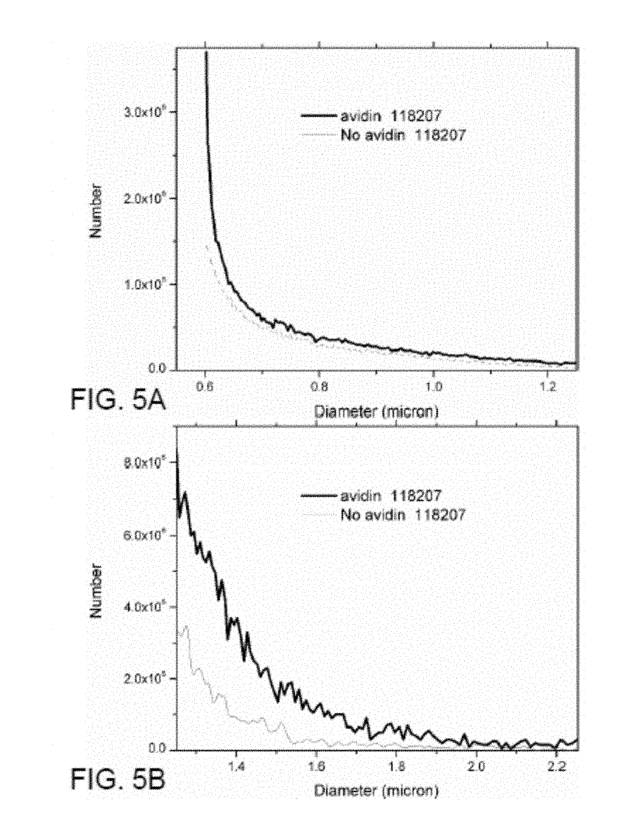

FIG. 5A is a graph showing the size distribution of droplets having a size of 0.6 microns to 1.25 microns. FIG. 5B is a graph showing the size distribution of droplets having a size of 1.25 microns to 2.25 microns. The thin line is for the droplet solution without added NeutrAvidin®. The thicker line is for the droplet solution incubated with NeutrAvidin®. As seen here, the number of particles of a given size was greater when NeutrAvidin® was added, or put another way the line was shifted to the right (e.g. greater particle sizes).

Polymeric particles may also be produced through a continuous and discontinuous phase emulsion where there is an aqueous phase and a discontinuous monomer phase. The reaction vessel for the emulsion may also contain surfactants and free radical initiators. As the emulsion is stirred, it is heated and free radical initiators are introduced into the emulsion. This causes the monomer particles to polymerize and thus gives a microparticles mixture of polymerized microparticles in the aqueous phase. This process allows for uniform size particles. An example of this process is styrene monomer dispersed in an aqueous phase with an octylphenol ethoxylate, a non-ionic surfactant, where benzoyl peroxide is introduced into the reaction vessel while the emulsion is stirred and heated.

Microparticles may also be produced using a technique of electro hydrodynamic spraying (ENDS) where a polymeric fluid is sprayed into a gas mixture such that the atomization of the liquid stream while it is being sprayed allows for very fine particle size generation. The polymer may be seated before it is introduced into the spray nozzle. Also, the polymer may be the reaction result of a dual or multicomponent mixture that is mixed prior to the spray nozzle and polymerizes as it moves through the spray nozzle and into the gas or gas mixture gas mixture. The gas may be an inert gas such as nitrogen or argon. The gas mixture may be air or other gas blends such as helium/oxygen and nitrogen/oxygen mixtures. The EHDS system is typically a physical process caused by the electric force applied to the surface of the liquid.

Microparticles and nanoparticles may also be produced by simple spray drying of a polymeric liquid or a polymeric liquid that is carried in an aqueous or solvent base.

The medium or primary fluid in which the particles are used may also be modified to increase the differentiation between the particles and the primary fluid.

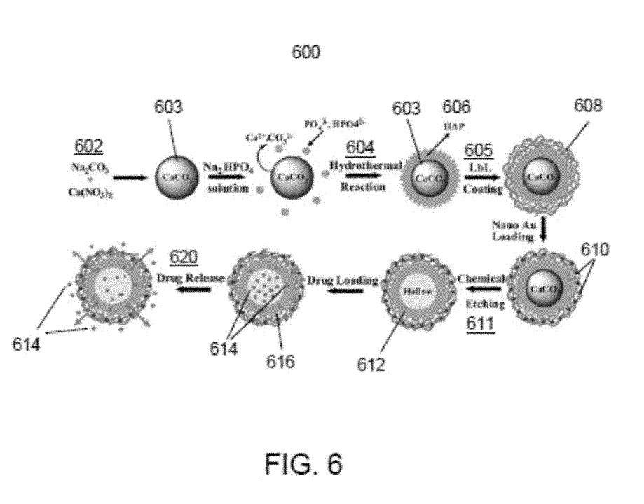

FIG. 6 illustrates a exemplary process 600 for creating and loading a payload into micro/nanoparticles and the release of that payload, described in more detail in Xu et al. “Hollow hierarchical hydroxyapatite/Au/polyelectrolyte hybrid microparticles for multi-responsive drug delivery,” J. Mater. Chem. B. 2014, 2, 6500-6507 which is herein incorporated by reference in its entirety. First at 602, Na₂CO₃ and Ca(NO₃)₂ are combined to form CaCo₃ template microparticles 603. Next, a (Ca10(PO4)6(OH)2, HAP) (“HAP”) coating 606 is applied to the CaCo₃ core 603 in a hydrothermal reaction at 604. HAP is used widely in the biomedical field due to its biocompatibility and biodegradability. Following the creation of the HAP layer 606, particles having the CaCo₃ core 603 and HAP coating 606 are then subject to a layer-by-layer (LbL) technique to incorporate polyectrolytes 608. Such polyelectrolytes include (aliphatic poly(urethane-amine)(PUA) and sodium poly(styrenesulfonate)(PSS). After the LbL coating 605, gold nanoparticles (AuNPs) 610, are loaded into the microparticles via electrostatic interaction. The AuNPs 610 help to slow the release of a payload loaded into a hollow particle.

A hollow HAP particle 612 is formed by removing the CaCo₃ core 603 with a chemical etching solution step 611, for example, acetic acid. The hollow HAP particle 612 is then loaded with payload 614 for payload delivery. Once the loaded particle 616 reaches a desired destination, the payload 614 may be released from the hollow particle carrier 612. Release/activation 620 of the payload 614 may be facilitated with a change in environmental temperature, pH, or in response to near infrared irradiation (NIR).

Devices and Systems

In general, the particles of the present disclosure may be manipulated with acoustic waves. The acoustic wave that may be utilized for manipulation of the microparticles and nanoparticles may be an acoustic standing wave such as a multidimensional acoustic standing wave, a planar standing wave, or combination of a multidimensional acoustic standing wave and a planar wave.

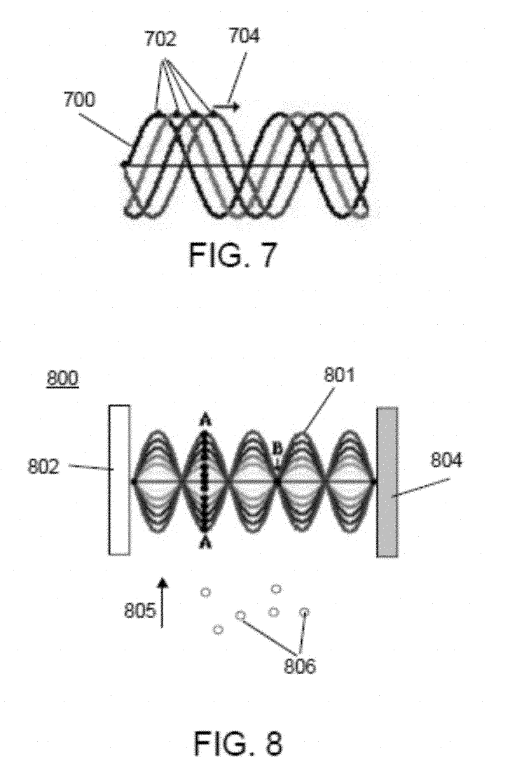

FIG. 7 illustrates an acoustic traveling wave 700. Acoustic waves are a type of longitudinal waves that propagate by means of adiabatic compression and decompression in a medium. The wave 700 includes a crest 702. The crest 702 moves in the direction of propagation 704.

An acoustic traveling wave 700 may change the contrast factor of the microparticles and nanoparticles when they are processed in an acoustic system. In other words, the contrast factor of the microparticles nanoparticles that are processed by a traveling acoustic wave may be different from the microparticles and nanoparticles when they are processed by an acoustic standing wave.

A combination of multiple traveling waves may generate an acoustic standing wave when each wave traveling in opposite directions creating a superposition of the waves. FIG. 8 illustrates an acoustic standing wave system 800 that creates an acoustic standing wave 801. The system is comprised of a reflector plate 804 and an ultrasonic transducer 802. Excitation frequencies typically in the range from hundreds of kHz to tens of MHz are applied by the transducer 802. One or more standing waves are created between the transducer 802 and the reflector 804. The standing wave is the sum of two propagating waves that are equal in frequency and intensity and that are traveling in opposite directions, e.g. from the transducer to the reflector and back. The propagating waves destructively interfere with each other and thus generate the standing wave. Point A on the medium moves from a maximum positive to a maximum negative displacement over time. The diagram only shows one-half cycle of the motion of the standing wave pattern. The motion would continue and persist, with point A returning to the same maximum positive displacement and then continuing its back-and-forth vibration between the up to the down position. Position A, having a maximum displacement is known as an anti-node. Note that point B on the medium is a point that never moves. Point B is a point of no displacement. Such points are known as nodes.

A fluid medium carrying particles 806 (microparticles or nanoparticles) disclosed above may flow in a direction 805 though an acoustic chamber/acoustic standing wave system 800. The standing wave 801 produced may trap the particles 806 against the fluid flow 805. Particles having a positive contrast factor would be trapped at a pressure node, while particles having a negative contrast factor would be trapped at an anti-node. Put another way, the particles are concentrated at a first location or a desired location. If the particles carry a payload, that payload may be released. That release may occur for example after passage of time (e.g. the shell dissolves or melts), or upon exposure to an outside energy source, or as previously described herein.

The acoustic devices discussed herein may operate in a multimode or planar mode. Multimode refers to generation of acoustic waves by an acoustic transducer that create acoustic forces in three dimensions. The multimode acoustic waves, which may be ultrasonic, are generated by one or more acoustic transducers, and are sometimes referred to herein as multi-dimensional or three-dimensional acoustic standing waves. Planar mode refers to generation of acoustic waves by an acoustic transducer that create acoustic forces substantially in one dimension, e.g. along the direction of propagation. Such acoustic waves, which may be ultrasonic, that are generated in planar mode are sometimes referred to herein as one-dimensional acoustic standing waves.

The acoustic devices may be used to generate bulk acoustic waves in a fluid/particle mixture. Bulk acoustic waves propagate through a volume of the fluid, and are different from surface acoustic waves which tend to operate at a surface of a transducer and do not propagate through a volume of a fluid.

The acoustic transducers may be composed of a piezoelectric material. Such acoustic transducers can be electrically excited to generate planar or multimode acoustic waves. The three-dimensional acoustic forces generated by multimode acoustic waves include radial or lateral forces that are unaligned with a direction of acoustic wave propagation. The lateral forces may act in two dimensions. The lateral forces are in addition to the axial forces in multimode acoustic waves, which are substantially aligned with the direction of acoustic wave propagation. The lateral forces can be of the same order of magnitude as the axial forces for such multimode acoustic waves. The acoustic transducer excited in multimode operation may exhibit a standing wave on its surface, thereby generating a multimode acoustic wave. The standing wave on the surface of the transducer may be related to the mode of operation of the multimode acoustic wave. When an acoustic transducer is electrically excited to generate planar acoustic waves, the surface of the transducer may exhibit a piston-like action, thereby generating a one-dimensional acoustic standing wave. Compared to planar acoustic waves, multimode acoustic waves exhibit significantly greater particle trapping activity on a continuous basis with the same input power. One or more acoustic transducers may be used to generate planar and/or multi-dimensional acoustic standing waves. In some modes of operations, multimode acoustic waves generate an interface effect that can hold back or retain particles of a certain size, while smaller particles can flow through the multimode acoustic waves. In some modes of operation, planar waves can be used to deflect particles at certain angles that are characteristic of the particle size.

Acoustophoresis is the separation of materials using acoustic waves. An implementation discussed herein provides a low-power, no-pressure-drop, no-clog, solid-state approach to particle separation from fluid dispersions. The scattering of the acoustic field off the particles creates secondary acoustic forces that draw particles together. The multimode operation results in a three-dimensional acoustic radiation force, which acts as a three-dimensional trapping field. The acoustic radiation force is proportional to the particle volume (e.g., the cube of the radius) when the particle is small relative to the wavelength. The acoustic radiation force is proportional to frequency and the acoustic contrast factor. The acoustic radiation force scales with acoustic energy (e.g., the square of the acoustic pressure amplitude). For harmonic excitation, the sinusoidal spatial variation of the force is what drives the particles to the stable positions within the standing waves. When the acoustic radiation force exerted on the particles is stronger than the combined effect of fluid drag force and buoyancy/gravitational force, the particle is trapped within the acoustic standing wave field. The action of the lateral and axial acoustic forces on the trapped particles results in formation of tightly packed clusters through concentration, clustering, clumping, agglomeration and/or coalescence of particles that, when reaching a critical size, settle continuously through enhanced gravity for particles heavier than the host fluid or rise out through enhanced buoyancy for particles lighter than the host fluid. Additionally, secondary inter-particle forces, such as Bjerkness forces, aid in particle agglomeration.

The following discussion is directed towards biological cells, which can be considered as particles for purposes of acoustophoretics. Most biological cell types present a higher density and lower compressibility than the fluid medium in which they are suspended, so that the acoustic contrast factor between the cells and the medium has a positive value. As a result, the axial acoustic radiation force (ARF) drives the cells towards the standing wave pressure nodes. The axial component of the acoustic radiation force drives the cells, with a positive contrast factor, to the pressure nodes, whereas cells or other particles with a negative contrast factor are driven to the anti-nodes. The radial or lateral component of the acoustic radiation force is the force that traps the cells. The radial or lateral component of the ARF is larger than the combined effect of fluid drag force and gravitational force.

For a cell to be trapped in the multi-dimensional ultrasonic standing wave, the force balance on the cell can be assumed to be zero, and, therefore, an expression for lateral acoustic radiation force F_{LRF} is F_{LRF}=F_{D}+F_{B}, where F_{D} is the drag force and F_{B} is the buoyancy force. For a cell of known size and material property, and for a given flow rate, this equation can be used to estimate the magnitude of the lateral acoustic radiation force.

One theoretical model that is used to calculate the acoustic radiation force is based on the formulation developed by Gor'kov. The primary acoustic radiation force F_{A} is defined as a function of a field potential U, F_{A}=−∇(U), which is affected by the acoustic pressure p, the fluid particle velocity u, the ratio of cell density ρₚ to fluid density ρ_{f}, the ratio of cell sound speed cₚ to fluid sound speed c_{f}, and the volume of the biological cell Vₒ.

Gor'kov's theory may be limited to particle sizes that are small with respect to the wavelength of the sound fields in the fluid and the particle, and it also may not take into account the effect of viscosity of the fluid and the particle on the radiation force. Additional theoretical and numerical models have been developed for the calculation of the acoustic radiation force for a particle without any restriction as to particle size relative to wavelength. These models also include the effect of fluid and particle viscosity, and therefore are a more accurate calculation of the acoustic radiation force. The models that were implemented are based on the theoretical work of Yurii llinskii and Evgenia Zabolotskaya as described in AIP Conference Proceedings, Vol. 1474-1, pp. 255-258 (2012). Additional in-house models have been developed to calculate acoustic trapping forces for cylindrical shaped objects, such as the “hockey pucks” of trapped particles in the standing wave, which closely resemble a cylinder.

Desirably, the ultrasonic transducer(s) generates a multi-dimensional standing wave in the fluid that exerts a lateral force on the suspended particles to accompany the axial force. Typical results published in literature state that the lateral force is two orders of magnitude smaller than the axial force. In contrast, the technology disclosed in this application provides for a lateral force to be of the same order of magnitude as the axial force. However, in certain embodiments described further herein, the device uses both transducers that produce multi-dimensional acoustic standing waves and transducers that produce planar acoustic standing waves. The lateral force component of the total acoustic radiation force (ARF) generated by the ultrasonic transducer(s) of the present disclosure is significant and is sufficient to overcome the fluid drag force at linear velocities of up to 1 cm/s, and to create tightly packed clusters, and is of the same order of magnitude as the axial force component of the total acoustic radiation force.

The acoustic standing wave is a three-dimensional acoustic field, which, in the case of excitation by a rectangular transducer, can be described as occupying a roughly rectangular prism volume of fluid. The transducer can be configured to face a reflector or boundary to permit generation of a standing wave therebetween. The transducer can be configured to face another transducer, both of which are operated to generate a standing wave therebetween. The transducer can be configured to face an acoustically absorbent material to permit generation of a traveling wave.

In some examples, the rectangular prism includes two opposing faces defined by the transducer and the reflector, an adjacent pair of opposing faces composed of the walls of the device, and a final opposing pair of faces that may define a flow channel entrance and exit. The acoustic wave generated by the transducer and the reflector create an interface or barrier region interface near the flow channel entrance, e.g., located near the upstream face of the acoustic standing wave field, generating an “acoustic barrier or edge effect”. This location is also referred to as an upstream interface region. The acoustic barrier can prevent particles with certain characteristics, such as a high acoustic contrast factor, for example, from passing through the acoustic wave generated by the transducer and the reflector.

The particles that are retained or blocked by the acoustic barrier may be captured in a chamber, such as a column, or returned to a holding device, such as a bioreactor. A circulating flow motion can be generated next to the acoustic barrier by a primary recirculation stream and can be optimized with acoustic chamber geometry variations to improve system efficiency.

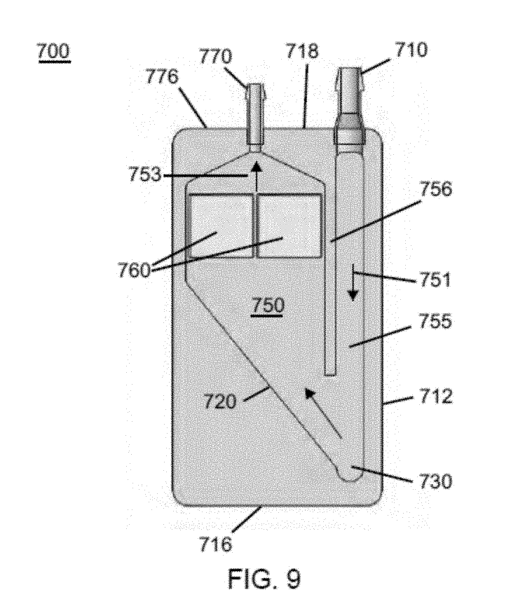

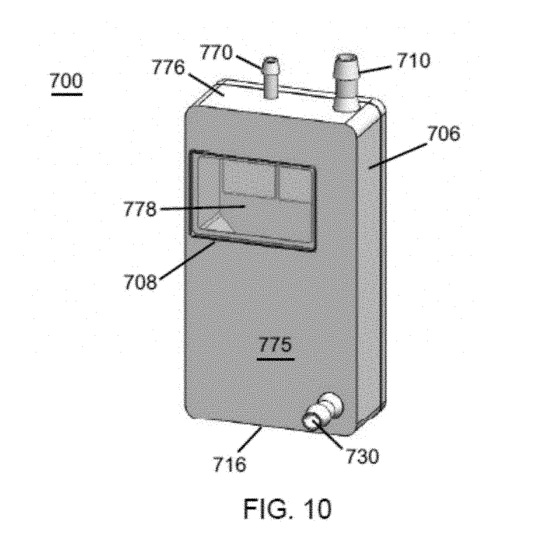

FIG. 9 and FIG. 10 are views of an acoustophoretic device that can be used with the particles of the present disclosure. FIG. 9 is a front cross-sectional view, and FIG. 10 is an exterior perspective view. Notably, this embodiment is specifically designed such that it can be fabricated with clean machining techniques, using Class VI materials (medical device grade HDPE, for example), or even as single or welded injection molded part. In this manner, this embodiment is an example of a single-use device, which is gamma-stable. The devices are flushed to remove bioburden and then gamma-irradiated (generally from 25-40 kGy) to sterilize any potential contamination that could destroy a healthy cell culture, such as that present in a perfusion bioreactor.

Referring first to FIG. 9, in this device 700, the inlet port 710 and the collection port 770 are both located at the top end 718 of the device, or on the top wall 776 of the device. The outlet port 730 is located at a bottom end 716 of the device. Here, the inlet port 710 and the outlet port 730 are both on a first side 712 of the device. The inlet flow path 751 is in the form of a channel 755 that runs from the inlet port downwards towards the bottom end and past the outlet port, the channel being separated from the acoustic chamber 750 (here, the separation occurring by an internal wall 756). Fluid will flow downwards in the channel, then rise upwards into the acoustic chamber 750. The bottom wall 720 of the acoustic chamber is a sloped planar surface that slopes down towards the outlet port 730. The location of the ultrasonic transducers 760 are shown here as two squares, between the top end and the bottom end of the device. The collection flow path 753 is located above the transducers.

Referring now to FIG. 10, the device 700 is shown as being formed within a three-dimensional rectangular housing 706. It can be seen that the outlet port 730 at the bottom end 716 of the device is located on a front wall 775. Again, the collection port 770 and the inlet port 710 are located on a top wall 776. A viewing window 708 made of a transparent material is present in the front wall. Through that viewing window, it can be seen that the ultrasonic transducers are mounted in the rear wall 778 of the device housing 706. The viewing window acts as a reflector to generate the multi-dimensional acoustic standing waves.

The device 700 can be used to cause cells and particles to react with each other, with the particles delivering payloads to the cells in roughly the area around the transducers 760, where acoustic waves are present. The cells can then exit through outlet port 730, while other fluid exits through collection port 770.

The particles can also interact with the cells and perform negative or positive selection depending upon the functionalization on the surface of the particle and the desired cells to be selected. The functionalized portion of the particle will bind with the receptors on the surface of the target cells such that the cells may be removed or retained in the system.

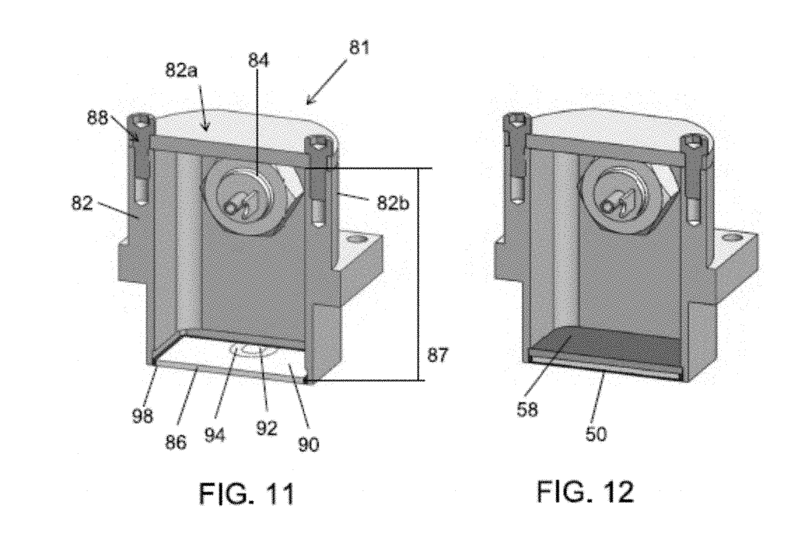

FIG. 11 is a cross-sectional view of an ultrasonic transducer 81 according to an example of the present disclosure, which is used in the acoustic filtering device of the present disclosure. Transducer 81 is shaped as a disc or a plate, and has an aluminum housing 82. The aluminum housing has a top end and a bottom end. The transducer housing may also be composed of plastics, such as medical grade HDPE or other metals. The piezoelectric element is a mass of perovskite ceramic, each consisting of a small, tetravalent metal ion, usually titanium or zirconium, in a lattice of larger, divalent metal ions, usually lead or barium, and O²⁻ ions. In this example, a PZT (lead zirconate titanate) piezoelectric element 86 defines the bottom end of the transducer, and is exposed from the exterior of the bottom end of the housing. The piezoelectric element is supported on its perimeter by a small elastic layer 98, e.g. epoxy, silicone or similar material, located between the piezoelectric element and the housing. Put another way, no wear plate or backing material is present. However, in some embodiments, there is a layer of plastic or other material separating the piezoelectric element from the fluid in which the acoustic standing wave is being generated. The piezoelectric element/crystal has an exterior surface (which is exposed) and an interior surface as well. In particular embodiments, the piezoelectric element/crystal is an irregular polygon, and in further embodiments is an asymmetrical irregular polygon.

Screws 88 attach an aluminum top plate 82a of the housing to the body 82b of the housing via threads. The top plate includes a connector 84 for powering the transducer. The top surface of the PZT piezoelectric element 86 is connected to a positive electrode 90 and a negative electrode 92, which are separated by an insulating material 94. The electrodes can be made from any conductive material, such as silver or nickel. Electrical power is provided to the PZT piezoelectric element 86 through the electrodes on the piezoelectric element. Note that the piezoelectric element 86 has no backing layer or epoxy layer. Put another way, there is an interior volume or an air gap 87 in the transducer between aluminum top plate 82a and the piezoelectric element 86 (e.g. the housing is empty). A minimal backing 58 (on the interior surface) and/or wear plate 50 (on the exterior surface) may be provided in some embodiments, as seen in FIG. 12.

The transducer design can affect performance of the system. A typical transducer is a layered structure with the ceramic piezoelectric element bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in one embodiment of the present disclosure, the transducers do not have a wear plate or backing, allowing the piezoelectric element to vibrate in one of its eigenmodes with a high Q-factor, or in a combination of several eigenmodes. The vibrating ceramic piezoelectric element/disk is directly exposed to the fluid flowing through the fluid cell.

Removing the backing (e.g. making the piezoelectric element air backed) also permits the ceramic piezoelectric element to vibrate at higher order modes of vibration with little damping (e.g. higher order modal displacement). In a transducer having a piezoelectric element with a backing, the piezoelectric element vibrates with a more uniform displacement, like a piston. Removing the backing allows the piezoelectric element to vibrate in a non-uniform displacement mode. The higher order the mode shape of the piezoelectric element, the more nodal lines the piezoelectric element has. The higher order modal displacement of the piezoelectric element creates more trapping lines, although the correlation of trapping line to node is not necessarily one to one, and driving the piezoelectric element at a higher frequency will not necessarily produce more trapping lines.

The reflector may be of a nonplanar type such as a faceted reflector. The reflector may also be another transducer that may have a planar or nonplanar surface. In some examples, two opposing transducers are used to generate an acoustic wave, such as an acoustic standing wave therebetween.

In some embodiments of the acoustic filtering device of the present disclosure, the piezoelectric element may have a backing that minimally affects the Q-factor of the piezoelectric element (e.g. less than 5%). The backing may be made of a substantially acoustically transparent material such as balsa wood, foam, or cork which allows the piezoelectric element to vibrate in a higher order mode shape and maintains a high Q-factor while still providing some mechanical support for the piezoelectric element. The backing layer may be a solid, or may be a lattice having holes through the layer, such that the lattice follows the nodes of the vibrating piezoelectric element in a particular higher order vibration mode, providing support at node locations while allowing the rest of the piezoelectric element to vibrate freely. The goal of the lattice work or acoustically transparent material is to provide support without lowering the Q-factor of the piezoelectric element or interfering with the excitation of a particular mode shape.

Placing the piezoelectric element in direct contact with the fluid also contributes to the high Q-factor by avoiding the dampening and energy absorption effects of the epoxy layer and the wear plate. Other embodiments of the transducer(s) may have wear plates or a wear surface to prevent the PZT, which contains lead, contacting the host fluid. This may be desirable in, for example, biological applications such as separating blood, biopharmaceutical perfusion, or fed-batch filtration of mammalian cells. Such applications might use a wear layer such as chrome, electrolytic nickel, or electroless nickel. Chemical vapor deposition may be used to apply a layer of poly(p-xylylene) (e.g. Parylene) or another polymer. Organic and biocompatible coatings such as silicone or polyurethane are also usable as a wear surface. Thin films, such as a PEEK film, can also be used as a cover of the transducer surface exposed to the fluid with the advantage of being a biocompatible material. In one embodiment, the PEEK film is adhered to the face of the piezo-material using pressure sensitive adhesive (PSA). Other films can be used as well.

In some embodiments, for applications such as oil/water emulsion splitting and others such as perfusion, the ultrasonic transducer has a nominal 2 MHz resonance frequency. Each transducer can consume about 28 W of power for droplet trapping at a flow rate of 3 GPM. This translates to an energy cost of 0.25 kW hr/m³. This is an indication of the very low cost of energy of this technology. Each transducer may be powered and controlled by a dedicated driver, which may include an amplifier, or multiple transducers may be driven by a single driver. In other embodiments, the ultrasonic transducer uses a square piezoelectric element, for example with 1″×1″ dimensions. Alternatively, the ultrasonic transducer can use a rectangular piezoelectric element, for example with 1″×2.5″ dimensions. Power dissipation per transducer was 10 W per 1″×1″ transducer cross-sectional area and per inch of acoustic standing wave span in order to get sufficient acoustic trapping forces. For a 4″ span of an intermediate scale system, each 1″×1″ square transducer consumes 40 W. The larger 1″×2.5″ rectangular transducer uses 100 W in an intermediate scale system. The array of three 1″×1″ square transducers would consume a total of 120 W and the array of two 1″×2.5″ transducers would consume about 200 W. Arrays of closely spaced transducers represent alternate potential embodiments of the technology. Transducer size, shape, number, and location can be varied as desired to generate desired multi-dimensional acoustic standing wave patterns.

The size, shape, and thickness of the transducer determine the transducer displacement at different frequencies of excitation, which in turn affects separation efficiency. Typically, the transducer is operated at frequencies near the thickness resonance frequency (half wavelength). Gradients in transducer displacement typically result in more trapping locations for the cells/biomolecules. Higher order modal displacements generate three-dimensional acoustic standing waves with strong gradients in the acoustic field in all directions, thereby creating equally strong acoustic radiation forces in all directions, leading to multiple trapping lines, where the number of trapping lines correlate with the particular mode shape of the transducer.

The lateral force of the acoustic radiation force generated by the transducer can be increased by driving the transducer in higher order mode shapes, as opposed to a form of vibration where the crystal effectively moves as a piston having a uniform displacement. The acoustic pressure is proportional to the driving voltage of the transducer. The electrical power is proportional to the square of the voltage. The transducer is typically a thin piezoelectric plate, with electric field in the z-axis and primary displacement in the z-axis. The transducer is typically coupled on one side by air (e.g., the air gap within the transducer) and on the other side by the fluid mixture of the cell culture media. The types of waves generated in the plate are known as composite waves. A subset of composite waves in the piezoelectric plate is similar to leaky symmetric (also referred to as compressional or extensional) Lamb waves. The piezoelectric nature of the plate typically results in the excitation of symmetric Lamb waves. The waves are leaky because they radiate into the water layer, which result in the generation of the acoustic standing waves in the water layer. Lamb waves exist in thin plates of infinite extent with stress free conditions on its surfaces. Because the transducers of this embodiment are finite in nature, the actual modal displacements are more complicated.

The transducers are driven so that the piezoelectric element vibrates in higher order modes of the general formula (m, n), where m and n are independently 1 or greater. Generally, the transducers will vibrate in higher order modes than (2,2). Higher order modes will produce more nodes and antinodes, result in three-dimensional standing waves in the water layer, characterized by strong gradients in the acoustic field in all directions, not only in the direction of the standing waves, but also in the lateral directions. As a consequence, the acoustic gradients result in stronger trapping forces in the lateral direction.

In embodiments, the voltage signal driving the transducer can have a sinusoidal, square, sawtooth, pulsed, or triangle waveform; and have a frequency of 50 kHz to 10 MHz. The voltage signal can be driven with pulse width modulation, which produces any desired waveform. The voltage signal can also have amplitude or frequency modulation start/stop capability to eliminate streaming.

The transducers are used to create a pressure field that generates acoustic radiation forces of the same order of magnitude both orthogonal to the standing wave direction and in the standing wave direction. When the forces are roughly the same order of magnitude, particles of size 0.1 microns to 300 microns will be moved more effectively towards “trapping lines”, so that the particles will not pass through the pressure field and continue to exit through the collection ports of the filtering device. Instead, the particles will remain within the acoustic chamber to be recycled back to the bioreactor.

In biological applications, all of the parts of the system (e.g., the bioreactor, acoustic filtering device, tubing fluidly connecting the same, etc.) can be separated from each other and be disposable. Acoustophoretic separators can provide improved performance over centrifuges and filters, by permitting better separation of the CHO cells without lowering the viability of the cells. The transducers may also be driven to create rapid pressure changes to prevent or clear blockages due to agglomeration of CHO cells. The frequency of the transducers may also be varied to obtain optimal effectiveness for a given power.

The techniques and implementations described herein may be used for integrated continuous automated bioprocessing. As a non-limiting example, CHO mAb processing may be carried out using the techniques and apparatuses described herein. Control can be distributed to some or all units involved in the bioprocessing. Feedback from units can be provided to permit overview of the bioprocess, which may be in the form of screen displays, control feedbacks, reporting, status reports and other information conveyance. Distributed processing permits a high degree of flexibility in achieving a desired process control, for example by coordinating steps among units and providing a batch executive control.

The bioprocessing can be achieved with commercially available components, and obtain 100% cell retention. Cell density can be controlled via an external cell bleed based on a capacitance signal. The perfusion device utilizing an acoustic wave system can be implemented with biocompatible materials, and may include gamma sterilized and single use components. The processing system also permits ultrasonic flow measurement, which is noninvasive, and is capable of operating with high viscosity fluids. The system can be implemented with single use sterile connectors and a simple graphical user interface (GUI) for control.

The acoustic wave system includes a sweeping flow that is induced below the acoustic chamber. An acoustic standing wave can act as a barrier for particulates in the fluid to permit a clarified stream to be passed and extracted. The recirculation loop can be implemented with high fluid velocity and with a low shear rate. The fluid velocity through the acoustic field can be lower than the fluid velocity through the recirculation loop, which may help to improve separation with low shear forces.

Positive and negative selection of cells may also be performed using various particles. For instance, the negative selection of TCR positive T cells is a process where functionalized particles bind with TCR positive T cells such that the TCR positive T cell is removed from the system. TCR positive T cells are deleterious to processes such as chimeric antigen receptor T cell therapies (CAR-T).

A positive selection process may also be utilized for specific cells where modified T-cells are selected by appropriately functionalized particles such that they are culled from a cell culture to then subsequently be utilized in a cellular therapy.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

1. A method for concentrating particles in a primary fluid at a first location, comprising:
flowing a fluid mixture comprising the particles and the primary fluid through an acoustophoretic device that comprises:
an acoustic chamber through which the fluid mixture flows; and
an ultrasonic transducer including a piezoelectric material that can be driven to create an acoustic wave in the acoustic chamber; and
driving the ultrasonic transducer to create the acoustic wave;
wherein the acoustic wave concentrates the particles at the first location.

2. The method of claim 1, wherein the acoustic wave is a multi-dimensional acoustic standing wave, a planar acoustic standing wave, a combination of a multi-dimensional acoustic standing wave and a planar acoustic standing wave, or an acoustic traveling wave.

3. The method of claim 1, wherein the particles contain a payload.

4. The method of claim 3, wherein the payload is a virus, a nucleic acid, a cytokine, a pharmaceutical molecule, a liquid, a gas, or mixtures thereof.

5. The method of claim 3, further comprising releasing the payload from the particles at the first location.

6. The method of claim 1, wherein the particles are solid, cellular, hollow, or a foam.

7. The method of claim 1, wherein the particles are made of one or more polymeric materials, ionomers, ceramics, or glass.

8. The method of claim 7, wherein the one or more polymeric materials are selected from the group consisting of polyethylene, polypropylene, polystyrene, divinylbenzene, poly methyl methacrylate, polysaccharide, polylactic acid (PLA), and poly(lactic-co-glycolic acid) (PLGA).

9. The method of claim 1, wherein the particles are formed from multiple layers of polymeric materials.

10. The method of claim 1, wherein the particles are hollow, and are made of glass, and have an ablative polymer coating an exterior surface of the glass.

11. The method of claim 10, wherein the ablative polymer is a polysaccharide that is functionalized with an antigen, antibody, or protein.

12. The method of claim 1, wherein the particles comprise:
a liquid core; and
a lipid shell encapsulating the liquid core.

13. The method of claim 12, wherein the liquid in the liquid core comprises a perfluorocarbon.

14. The method of claim 13, wherein the perfluorocarbon is perfluoropentane, perfluorohexane, perfluorooctane, perfluorooctyl bromide, perfluorodichlorooctane, or perfluorodecalin.

15. The method of claim 12, wherein the lipid shell is formed from dipalmitoylphosphatidylcholine (DPPC), 1,2-palmitoyl-phosphatidic acid (DPPA), a lipid-polyethylene glycol conjugate, or a complex of a lipid with albumin.

16. The method of claim 12, wherein the lipid shell is functionalized with streptavidin, biotin, avidin, or an antibody.

17. A particle, comprising:
a liquid core; and
a lipid shell encapsulating the liquid core.

18. The particle of claim 17, wherein the liquid in the liquid core comprises a perfluorocarbon.

19. The particle of claim 18, wherein the perfluorocarbon is perfluoropentane, perfluorohexane, perfluorooctane, perfluorooctyl bromide, perfluorodichlorooctane, or perfluorodecalin.

20. The particle of claim 17, wherein the lipid shell is formed from dipalmitoylphosphatidylcholine (DPPC), 1,2-palmitoyl-phosphatidic acid (DPPA), a lipid-polyethylene glycol conjugate, or a complex of a lipid with albumin.

21. The particle of claim 17, wherein the lipid shell is functionalized with streptavidin, biotin, avidin, or an antibody.

22. A method for separating target particles from a fluid, comprising:
receiving functionalized particles in the fluid in a chamber;
receiving target particles in the chamber;
permitting the target particles to bind with the functionalized particles;
applying an acoustic wave to the chamber to influence the functionalized particles to be collected or blocked by the acoustic wave.

23. The method of claim 22, wherein the functionalized particles comprise a perfluorocarbon that is one or more of perfluoropentane, perfluorohexane, perfluorooctane, perfluorooctyl bromide, perfluorodichlorooctane, or perfluorodecalin
